(19) United States
(12) Patent Application Publication
(10) Pub. No.: US 2017/0246437 A1
(43) Pub. Date: Aug. 31, 2017

(54) pH-MODULATING DRUG DELIVERY DEVICES AND METHODS

(71) Applicant: TARIS Biomedical LLC, Lexington, MA (US)

(72) Inventor: Heejin Lee, Bedford, MA (US)

(21) Appl. No.: 15/510,558

(22) PCT Filed: Sep. 11, 2015
(86) PCT No.: PCT/US2015/049841

§ 371 Date: Mar. 10, 2017

Related U.S. Application Data
(60) Provisional application No. 62/049,991, filed on Sep. 12, 2014.
(60) Provisional application No. 62/054,748, filed on Sep. 24, 2014.

(51) Int. Cl. A61M 31/00; A61K 47/34; A61K 9/00

(52) U.S. Cl. CPC A61M 31/002; A61K 9/0092; A61K 9/0034; A61K 47/34

(57) ABSTRACT

Drug delivery devices and methods are provided for modulating the microenvironmental pH surrounding the device. One embodiment of a drug delivery device includes an elongated annular body formed of a matrix system containing a drug dispersed in a biocompatible polymer and a pH modifier disposed in the lumen of the body, wherein the device is configured to release in vivo (i) the drug by diffusion from the annular body and (ii) the pH modifier by diffusion through the annular body. Methods in clude inserting into the patient a device and releasing the drug and pH modifier therefrom by diffusion.

### CROSS-REFERENCE TO RELATED APPLICATIONS

This application claims priority benefit of U.S. Provisional Application No. 62/049,991, filed Sep. 12, 2014, and of U.S. Provisional Application No. 62/054,748, filed Sep. 24, 2014, both of which are incorporated by reference herein.

### BACKGROUND

The present disclosure relates generally to the field of implantable drug delivery devices, and more particularly to devices for modulating the microenvironmental pH surrounding the device.

Systems for administering a drug to a patient from a device deployed in vivo within the patient, e.g., an implantable drug delivery device, are known. The release kinetics of some drug delivery systems may depend on the pH of the fluid at the tissue site into which the drug is to be released. For example, drugs with pH-dependent solubility, such as weakly acidic or weakly basic drugs, have been shown to exhibit pH-dependent drug release. This characteristic of the drug can lead to unwanted variability in the amounts or rates of drug delivery. The pH at a given tissue site may vary over time within a patient and/or may vary among different patients. This can lead to unacceptable variability in drug release kinetics and dosing.

Numerous and varied efforts have been made to achieve pH-independent drug release. However, most of these efforts relate to oral dosage forms and formulating such drug tablets with various excipients that are mixed with the drug. It therefore would be desirable to reduce or minimize the pH-dependent behavior of drugs by providing release systems that can lower the inter- and intra-patient variability.

Devices for local drug delivery to the bladder are known. For example, U.S. Pat. No. 8,679,094, which is incorporated herein by reference, describes intravesical devices for drug delivery that are deployable into the bladder of a patient and are well tolerated by the patient. The composition of urine in the bladder in which the device is deployed may, however, have a pH that is suboptimal for the release of particular therapeutic agents from the device. It therefore would also be desirable to provide new and different drug delivery device designs and/or methods for improving the release of the therapeutic agents into such suboptimal pH environments.

### SUMMARY

In one aspect, drug delivery devices are provided, including an elongated annular body having a first end, an opposed second end, and a lumen extending between the first and second ends. In one embodiment, the elongated annular body is formed of a matrix system containing a drug dispersed in a biocompatible polymer, a pH modifier is disposed in the lumen, and the device is configured to release in vivo (i) the drug by diffusion from the annular body and (ii) the pH modifier by diffusion from the lumen through the annular body. In another embodiment, the elongated annular body is formed of a biocompatible polymer, a drug and a pH modifier are disposed in the lumen, the device is configured to release in vivo the drug and the pH modifier by diffusion from the lumen through the annular body, and the device is configured to release the pH modifier in an amount effective to cause a proximal media pH adjacent an outer surface of the annular body to be dominated by a microenvironment pH created by the diffusion of the pH modifier and not by a macro pH of an external media.

In another aspect, methods of administering a drug to a patient in need thereof are provided, including (i) inserting into the patient a device including an elongated annular body formed of a matrix system containing a drug dispersed in a biocompatible polymer and having a first end, an opposed second end, and a lumen extending between the first and second ends, and a pH modifier disposed in the lumen, (ii) releasing the drug by diffusion from the annular body to the body of the patient, (iii) and releasing the pH modifier by diffusion from the lumen through the annular body to the body of the patient.

In yet another aspect, methods of administering a drug to a patient in need thereof are provided, including (i) inserting into the patient a device including an elongated annular body formed of a biocompatible polymer and having a first end, an opposed second end, and a lumen extending between the first and second ends, and a drug and a pH modifier disposed in the lumen, and (ii) releasing the drug and the pH modifier by diffusion from the lumen through the annular body, wherein the pH modifier is released in an amount effective to cause a proximal media pH adjacent an outer surface of the annular body to be dominated by a microenvironment pH created by the diffusion of the pH modifier and not by a macro pH of an external media.

### BRIEF DESCRIPTION OF THE DRAWINGS

Referring now to the drawings, which are meant to be exemplary and not limiting, and wherein like elements are numbered alike. The detailed description is set forth with reference to the accompanying drawings illustrating examples of the disclosure, in which use of the same reference numerals indicates similar or identical items. Certain embodiments of the present disclosure may include elements, components, and/or configurations other than those illustrated in the drawings, and some of the elements, components, and/or configurations illustrated in the drawings may not be present in certain embodiments. The drawings are not to scale.

FIG. 1A is a cross-sectional plan view of one embodiment of a drug delivery device.

FIG. 1B is a cross-sectional end view of the drug delivery device of FIG. 1A.

FIG. 1C is a partial cross-sectional perspective view of the drug delivery device of FIG. 1A.

FIG. 2 is a partial cross-sectional perspective view of one embodiment of a drug delivery device.

FIG. 3A is a cross-sectional plan view of one embodiment of a drug delivery device.

FIG. 3B is a cross-sectional end view of the drug delivery device of FIG. 3A.

FIG. 4A is a cross-sectional plan view of one embodiment of a drug delivery device.

FIG. 4B is a cross-sectional end view of the drug delivery device of FIG. 4A.

FIG. 5A is a cross-sectional plan view of one embodiment of a drug delivery device.

FIG. 5B is a cross-sectional end view of the drug delivery device of FIG. 5A.

FIG. 6 is a cross-sectional plan view of one embodiment of a drug delivery device.

FIG. 7 is a cross-sectional plan view of one embodiment of a drug delivery device.

FIG. 8 is a cross-sectional plan view of one embodiment of a drug delivery device.

FIG. 9 is a cross-sectional plan view of one embodiment of a drug delivery device.

FIG. 10 is a cross-sectional plan view of one embodiment of a drug delivery device.

FIG. 11 is a cross-sectional plan view of one embodiment of a drug delivery device.

FIG. 12 is a cross-sectional plan view of one embodiment of a drug delivery device.

FIG. 13 is a cross-sectional plan view of one embodiment of a drug delivery device.

FIG. 14 is a cross-sectional plan view of one embodiment of a drug delivery device.

FIG. 15 illustrates one embodiment of a drug delivery device that is elastically deformable between a straightened deployment shape and a coiled retention shape.

FIG. 16A illustrates one embodiment of a drug delivery device implanted into the bladder of a patient.

FIG. 16B is an exploded section view of the device of FIG. 16A.

### DETAILED DESCRIPTION

Drug delivery devices and methods have been developed for reducing the pH dependency of drug release by modulating the microenvironmental pH surrounding the device. As used herein, the term “drug” and “active pharmaceutical ingredient (API)” are used interchangeably.

In one aspect, drug delivery devices are provided, which include an elongated annular body having a first end, an opposed second end, and a lumen that extends between the first and second ends. As will be explained in more detail herein, the elongated annular body may be formed of a biocompatible polymer or a matrix system containing a drug dispersed in a biocompatible polymer. A pH modifier is disposed in the lumen of the annular body such that, in vivo, the pH modifier is released by diffusion from the lumen, through the annular body. For example, the annular body may be permeable to the pH modifier and/or the annular body may include one or more mechanically formed through-holes to provide a route for the pH modifier to contact biological fluids and to become solubilized and then be released from the device.

A drug may be dispersed in a matrix material forming the annular body and/or may be disposed in the lumen with the pH modifier (e.g., as a separate or integrated component), such that, in vivo, the drug is released by diffusion from the matrix material of the annular body and/or by diffusion from the lumen through the annular body. For example, when the drug is disposed in the lumen of the annular body, the material forming the annular body may be permeable to the drug and/or the annular body may include one or more mechanically formed through-holes to provide a route for the drug to contact biological fluids and to become solubilized and then released from the device.

In certain embodiments, the device is configured to release the pH modifier in an amount effective to cause a proximal media pH adjacent an outer surface of the annular body to be dominated by a microenvironment pH created by the diffusion of the pH modifier and not by a macro pH of an external media, such as biological fluids at the implantation site. Thus, the devices disclosed herein may modulate the immediately local pH surrounding the device, and thereby reduce or minimize the pH-dependent behavior of the drug released from the device by lowering the inter- and intra-patient variability of pH at the implantation microenvironment.

In various embodiments, the devices may further include a rate controlling layer disposed between the pH modifier and the annular body, which lowers the diffusion rate of the pH modifier from the device and thereby extends the duration of pH modifying effect.

In various embodiments, the devices may further include a drug-free layer disposed on an external surface of the annular body, which prevents direct contact between the annular body and surrounding tissue during in vivo use of the device.

Features and embodiments of such drug delivery devices and methods for their use are described in more detail below, with reference to the accompanying Figures.

Drug Delivery Devices

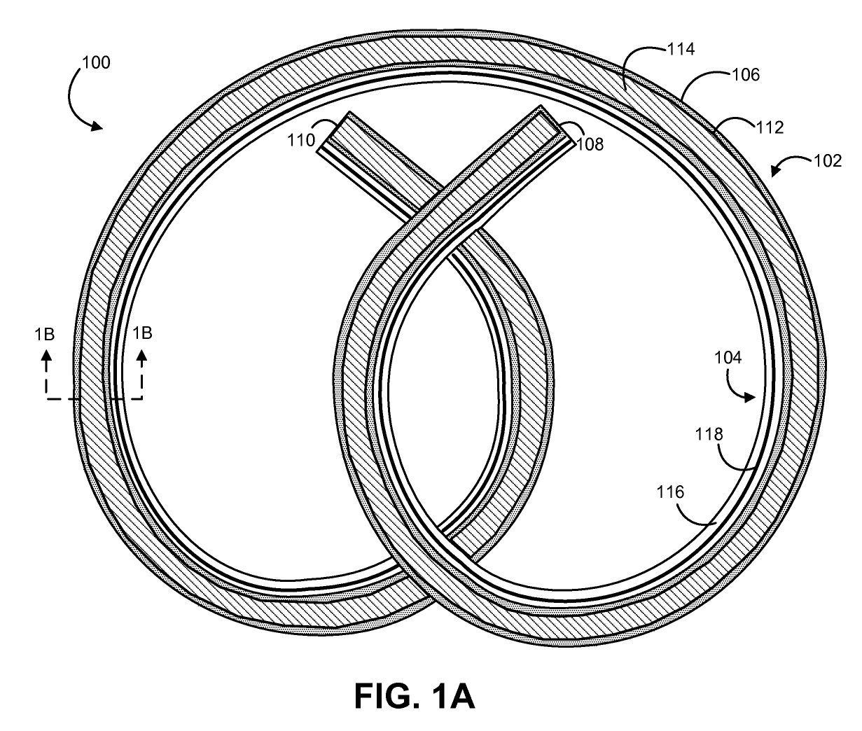

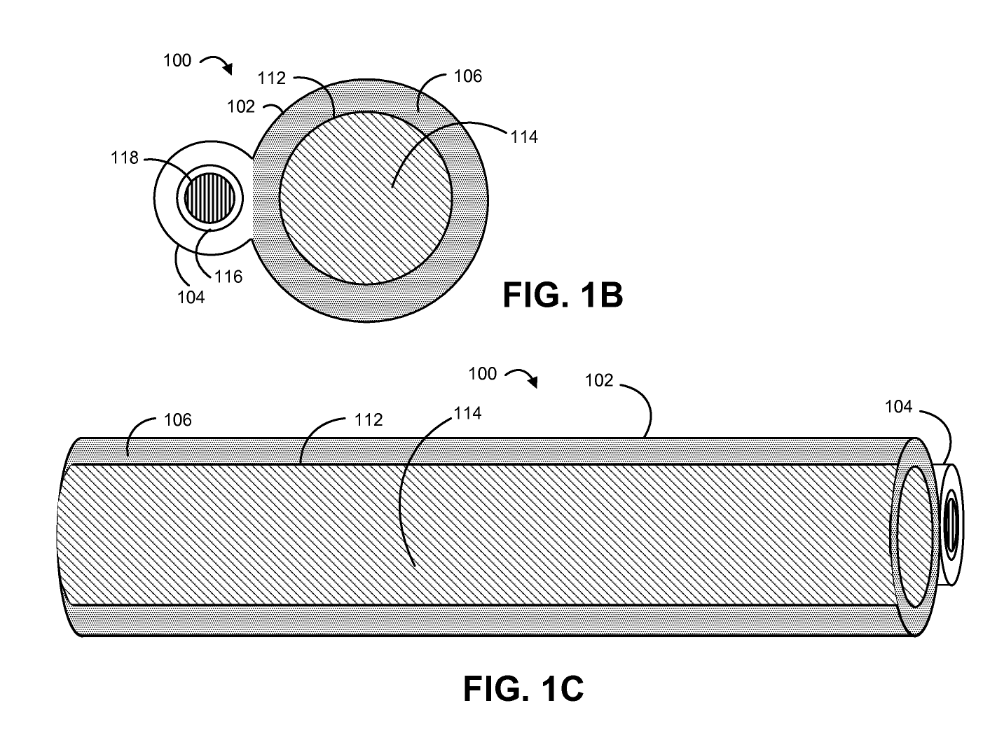

In certain embodiments, as shown in FIGS. 1A-1C, a drug delivery device 100 includes (i) an elongated annular body 106 formed of a matrix system containing a drug dispersed in a biocompatible polymer and having a first end 108, an opposed second end 110, and a lumen 112 extending between the first and second ends 106, 108, and (ii) a pH modifier 114 disposed in the lumen 112, wherein the device 100 is configured to release in vivo the drug by diffusion from the annular body 106 and the pH modifier 114 by diffusion from the lumen 112 through the annular body 106.

In these embodiments, pH modulation is advantageously achieved without needing to (re)formulate the drug or the drug/matrix material of the device to further include the pH modifying agent, since the drug and pH modifier are disposed in separate locations in the device. Furthermore, in these embodiments, the devices are configured such that a majority of the drug remains within an area proximal to the outer surface of the device body, to minimize the distances the drug must pass (e.g., by diffusion) for release of the drug from the device. In other words, these embodiments of the devices are configured to reduce or minimize the distances between the drug and the outer surface of the device. This feature may be particularly beneficial for releasing low solubility drugs at therapeutically effective rates following in vivo insertion.

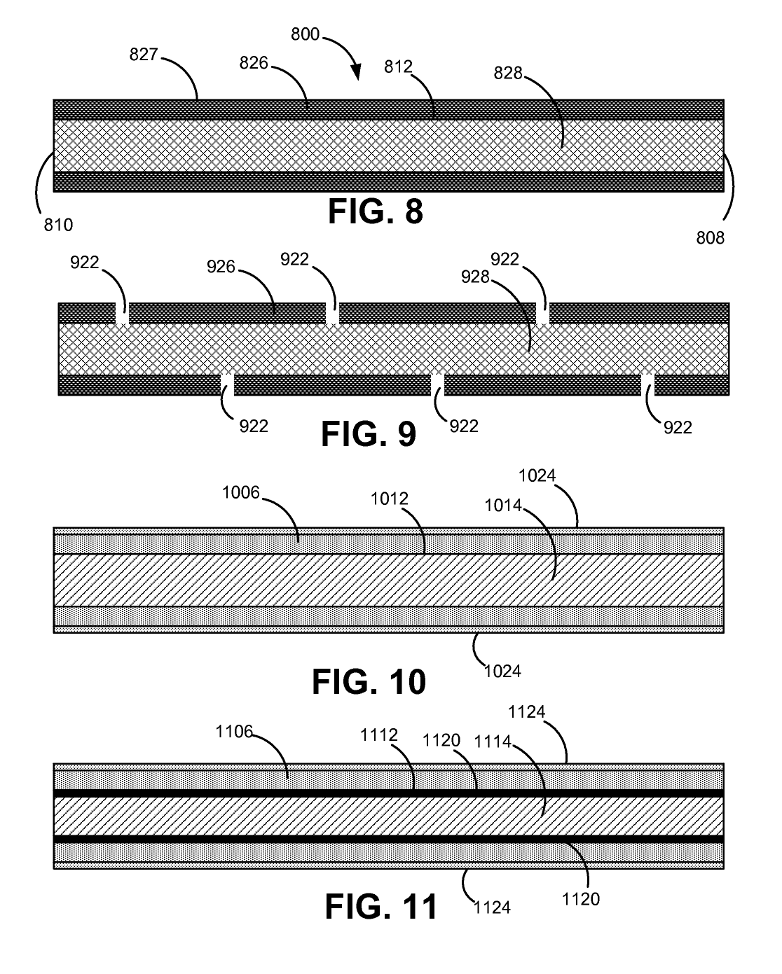

In other embodiments, as shown in FIG. 8, a drug delivery device 800 includes (i) an elongated annular body 826 formed of a biocompatible polymer and having a first end 808, an opposed second end 810, and a lumen 812 extending between the first and second ends 808, 810, and (ii) a drug and a pH modifier (shown collectively as 828, although the drug and pH modifier may also be separate components) disposed in the lumen 812, wherein the device is configured to release in vivo the drug and the pH modifier 828 by diffusion from the lumen 812 through the annular body 826, and wherein the device 800 is configured to release the pH modifier in an amount effective to cause a proximal media pH adjacent an outer surface 827 of the annular body to be dominated by a microenvironment pH created by the diffusion of the pH modifier and not by a macro pH of an external media.

Various embodiments and features of such devices will be described with reference to the Figures, in which the drug delivery devices are illustrated as having a tubular construction. However, the constructions disclosed herein can be readily adapted into non-tubular/non-annular configurations. For example, the elongated body, when viewed in cross-section, may be circular, substantially circular, non-circular, or have other configurations to increase the surface area of the sidewall(s) between the opposed ends of the body.

In certain embodiments, the elongated body has a length of from about 10 cm to about 20 cm. In certain embodiments, the terminal ends of the device may be sealed or otherwise closed, such as with an adhesive or other sealant, a plug or other closure.

Biocompatible Polymers and Matrix Systems

The biocompatible polymer of the device body may be any suitable biocompatible polymer, such as silicone (e.g., a low durometer silicone), ethylene vinyl acetate (EVA), or other non-degradable or degradable polymers. As used herein, the term “low durometer” refers to a material having a Shore hardness of 70 A or less, such as from 50 A to 70 A. As used herein, the term “high durometer” refers to a material having a Shore hardness of greater than 70 A, such as from 80 A to 65 D.

For embodiments, such as shown in FIGS. 1A-1C, in which the device body 106 is a matrix system containing the drug dispersed in the biocompatible polymer, the polymer should be chemically compatible with the drug, such that the desired diffusion profile of drug from the matrix is achieved. The matrix system may consist only of the drug with the biocompatible polymeric matrix material, or one or more pharmaceutically acceptable excipients may be included with the drug in the biocompatible polymeric matrix material.

The amount of drug in the polymeric matrix can vary. In one embodiment, the drug is present in the matrix in an amount from about 0.1 percent to about 40 percent, by weight. For example, the drug may be present in an amount of between 1 percent and 20 percent, or between 5 percent and 15 percent, by weight of the matrix. Lesser or greater amounts of drug are also possible, depending, for example, on the particular drug and polymer selected and on the desired drug release rate.

In certain embodiments, the drug-polymer matrix is formed by using conventional molding equipment and processes, e.g., injection molding. In one embodiment, the mold may be coil shaped. The molded matrix may be formed with or without a lumen for a retention frame depending on whether one is needed, as discussed in more detail below. In another embodiment, the drug polymer matrix is formed by an extrusion process, for example, wherein the matrix has a lumen for subsequent loading of a retention frame. In yet another embodiment, as shown in FIGS. 1A-1C, the device 100 body is formed by a co-extrusion process, with drug matrix portion 102 and an integral retention frame portion 104. For example, the drug matrix portion 102 may include a first (e.g., low durometer) silicone and the integral retention frame portion 104 may include a second (e.g., high durometer) silicone.

pH Modifiers

Any suitable pH adjusting agents may be used as the pH modifier. Examples of suitable pH modifiers include acetic acid, ammonium carbonate, ammonium hydroxide, ammonium phosphate, boric acid, fumaric acid, tartaric acid, succinic acid, citric acid, adipic acid, ascorbic acid, malic acid, nitric acid, sorbic acid, propionic acid, sulfuric acid, trolamine, diethanolamine, hydrochloric acid, L-cysteine hydrochloride, glycine hydrochloride, sodium acetate, magnesium oxide, magnesium hydroxide, magnesium trisilicate, dicalcium phosphate, sodium carbonate, sodium bicarbonate, sodium borate, sodium chloride, sodium glycolate, sodium hydroxide, sodium lactate, sodium propionate, sodium phosphate, sodium phosphate dibasic, sodium citrate, sodium citrate dehydrate, bentonite, arginine, disodium phosphate, monosodium phosphate, disodium phosphate, trisodium phosphate, sodium hydroxide, sodium lactate, potassium acetate, potassium chloride, potassium citrate, potassium hydroxide, potassium bicarbonate, potassium metaphosphate, potassium phosphate, potassium phosphate dibasic and monobasic, potassium citrate tribasic monohydrate, calcium acetate, calcium carbonate, sodium alginate, Carbopol and enteric polymers, and 2-amino-2methyl-1,3-propanediol (AMPD), and Eudragit® E100. In certain embodiments, the pH modifier is a carbonate, a citrate, or any combination thereof.

In some embodiments, the pH modifier can be a drug itself that has a sufficient pH modifying capability, such as an HCl salt form of a drug.

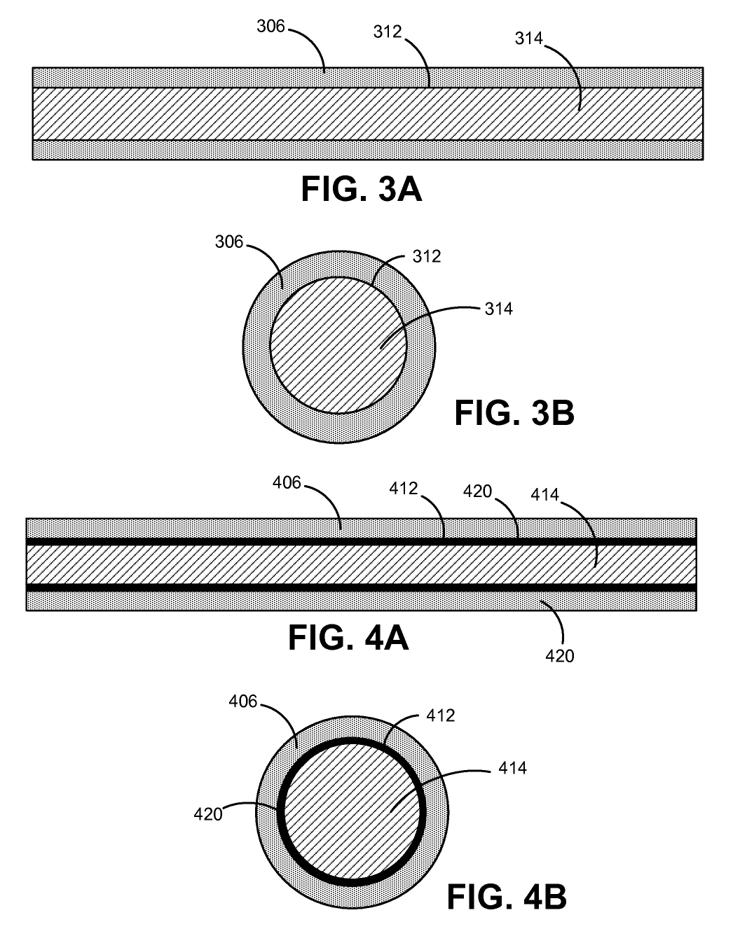

As shown in FIGS. 3A-3B, the matrix material of the body 306 and the pH modifier 314 may be selected such that the pH modifier diffuses through the matrix material 306 from the lumen 312. For example, the matrix system may provide an interstitial route for biological fluids to contact the pH modifier such that it becomes solubilized and then released from the lumen through the matrix system.

In certain embodiments, the pH modifier diffuses from the device at a rate that is sufficient to alter or adjust the microenvironment pH in the immediate vicinity surrounding the outer wall of the device. In such embodiments, the proximal media pH neighboring the outer wall will be dominated by the microenvironment pH created by pH modifier diffusion, and not by the macro pH of the external media, such as the bulk of the surrounding biological fluid.

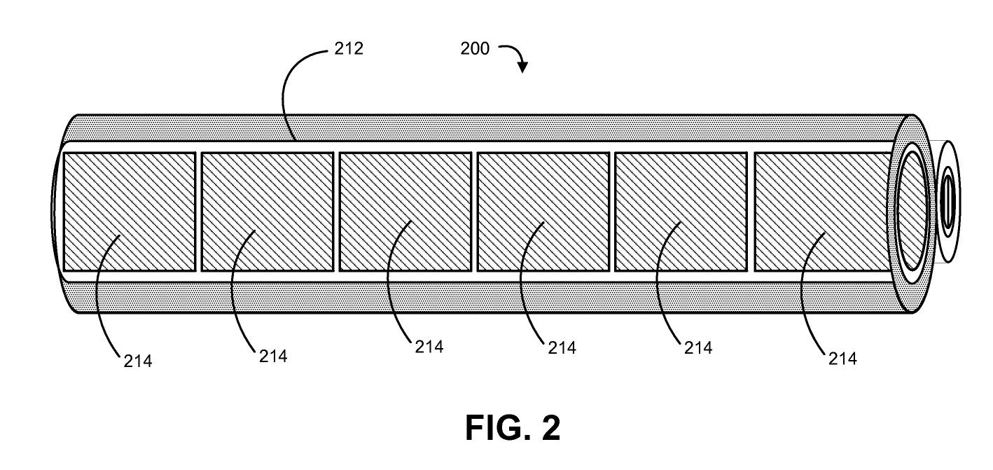

In certain embodiments, as shown in FIG. 2, the device 200 includes pH modifier in the form of tablets 214 disposed in the lumen 212 formed by the annular body 206. In embodiments, in which the drug disposed in the lumen of the device, the drug may also be in the form of one or more tablets. For example, the drug tablets may be distinct from the pH modifier tablets and/or the tablets may contain a mixture of the drug and the pH modifier (or the pH modifier may itself be a drug, as discussed above). Tablets 214 may also be drug-free.

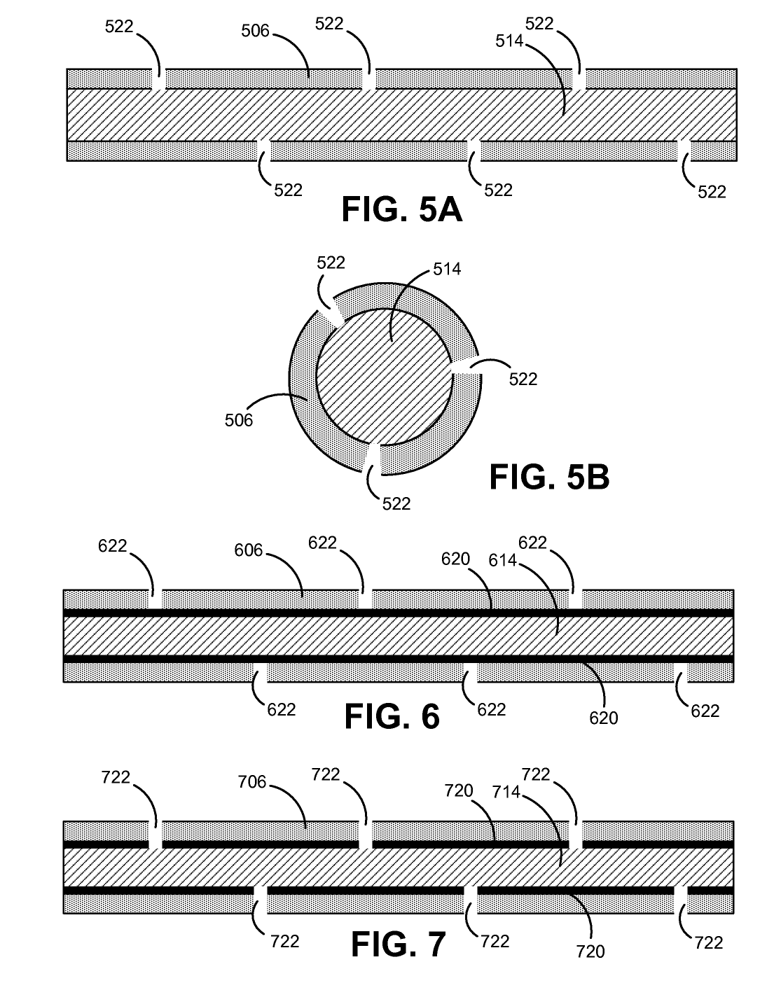

In certain embodiments, as shown in FIGS. 5A-5B, the pH modifier 514 cannot diffuse through the tubular matrix wall 506, either at all or at an effective rate. In such embodiments, one or more through-holes 522 may be distributed around the annular body wall 206. The number, size and position/layout of the holes will be determined by the pH altering strength of pH modifier, external media pH range to be adjusted, and/or API release rate from the outer tubular wall. For example, in some embodiments, the release of pH modifier from a single hole may not alter or adjust the microenvironment pH of the whole surface of the outer tube wall. Therefore, in one embodiment, as shown in FIGS. 5A-5B, multiple holes 522 are distributed around outer tube surface to provide pH modification over the entire outer tube surface.

As used herein the term “through-hole” refers to an aperture that is mechanically formed (e.g., laser drilled, molded, punched) and that is distinct from pores in a porous material.

Drug Form & Other Features

The drug may be dispersed in the matrix, or loaded into the lumen, in a variety of forms. It may be in powder or granule form, for example. In the lumen, the drug and the pH modifier may be provided as discrete materials, such as tablets, or they may be blended or otherwise formulated together. The drug and biocompatible polymer may be mixed together using any suitable process and equipment known in the art. The drug may be dispersed in the biocompatible polymer homogeneously or heterogeneously.

Any suitable drug may be used in the drug delivery device disclosed herein. Particular drugs that may be used in the present devices are discussed below, in relation to particular applications of the drug delivery devices.

In some embodiments, the drug is a high solubility drug. As used herein, the term “high solubility” refers to a drug having a solubility above about 10 mg/mL water at 37° C. In other embodiments, the drug is a low solubility drug. As used herein, the term “low solubility” refers to a drug having a solubility from about 0.01 mg/mL to about 10 mg/mL water at 37° C. The solubility of the drug may be affected at least in part by its form. For example, a drug in the form of a water soluble salt may have a high solubility, while the same drug in base form may have a low solubility.

In certain embodiments, as shown in FIG. 8, the API is not incorporated in the tube wall, and the API release mechanism is diffusion through the tube wall 826. The pH modifier resides in the tube with API (collectively 828). In this case, pH modifier can diffuse through the tubular housing wall as API can. In one embodiment, the API is its own pH modifier, providing a self-buffering action if its pH altering strength is enough to create the microenvironment pH.

In certain embodiments, as shown in FIG. 9, the drug and/or the pH modifier 928 cannot diffuse through the polymer device wall 926, either at all or at an effective rate. In such embodiments, one or more through-holes 922 may be distributed around the annular body wall 926. The number, size and position/layout of the holes will be determined by the pH altering strength of pH modifier, external media pH range to be adjusted, and/or desired API release rate.

If the drug is weak base and if drug release through the tube wall is dominated by the diffusion of its neutral (unionized) form only, drug release will be faster at lower media pH, especially at pH<<pKa of the drug, because the drug form will be mostly ionized one at such low pH, which helps maintain a low concentration of the neutral drug form (higher driving force for the diffusion of neutral form of the drug). Conversely, the drug will be slower at higher media pH, especially at pH>>pKa of the drug.

If the drug is weak acid and if drug release through the tube wall is dominated by the diffusion of its neutral (unionized) form only, drug release will be faster at higher media pH, especially at pH>>pKa of the drug, because the drug form will be mostly ionized one at such high pH, which helps maintain a low concentration of the neutral drug form (higher driving force for the diffusion of neutral form of the drug). Conversely, the drug will be slower at lower media pH, especially at pH<<pKa of the drug.

If the release rate of pH modifier from the device is sufficient to create microenvironment pH, which could be either acidic or alkaline, API release by diffusion through or from the body wall can be less or minimally dependent on the macro media pH. For example, consider a weak base drug with pH-dependent solubility that shows higher drug release at lower pH. If a basic pH modifier is chosen to create a high microenvironmental pH, then the weak base drug release rate from the device will not increase as much in acidic media as compared with the case without pH modifier. Conversely, if an acidic pH modifier is chosen to create a low microenvironmental pH, then the weak base drug release rate from the device will not decrease as much in alkaline media as compared with the case without a pH modifier.

Thus, the drug and pH modifier may be selected to achieve the desired microenvironmental pH for the desired drug delivery rate. The release rate of the drug from the device generally is controlled by the design of the combination of the device components, including but not limited to the materials, dimensions, surface area, and apertures of the annular body, and the drug-free and rate controlling layers, if any, as well as the particular drug and total mass and percentage of drug load in the system, among others.

Rate Controlling Layer

In some embodiments, as shown in FIGS. 4A-4B, the device further includes a rate controlling layer 420 disposed in the lumen 412 between the pH modifier 414 and the matrix system 406. For example, the rate controlling layer may lower the diffusion rate of the pH modifier to extend the duration of pH modifying effect, if needed. In certain embodiments, as shown in FIGS. 4A-4B, the pH modifier 414 can diffuse through both the rate controlling layer 420 and the tubular matrix wall 406. The rate controlling layer may be provided in the form of a coating.

For example, the rate controlling layer may be formed of ethylcellulose with a permeability modifier (i.e., pore former), such as SURELEASE® (manufactured by COLORCON, England), hydroxypropyl methylcellulose (HPMC), or metacrylic acid or methacrylate copolymers, such as EUDRAGIT® (manufactured by Evonik Nutrition & Care GmbH, Germany).

In one embodiment, the rate controlling layer is made of one or more polymers. For example, the rate controlling layer may be a hydrophilic polymer. In one embodiment, the rate controlling layer has a porous structure in order to provide a controlled diffusion path for the pH modifier. In other embodiments, the rate controlling layer has a non-porous structure. In some embodiments in which through-holes are provided in the annular body wall, those through-holes also extend through the rate controlling layer.

In certain embodiments, as shown in FIG. 6, the material of the rate controlling layer 620 is chosen so that the pH modifier 614 can diffuse through the layer 620, which is position inside the perforated (i.e., containing through-holes 622) API incorporated matrix tube wall 606. In another embodiment, as shown in FIG. 7, for example, such as when the pH modifier 714 cannot diffuse through the rate controlling layer 720, the rate controlling layer 720 is provided with one or more through-holes 722 and the matrix wall 706 is provided with one or more through-holes. The through-holes may or may not be aligned with any through-hole(s) in the matrix tube wall and/or with any through-holes in the drug-free layer discussed below.

Drug-Free Layer

In certain embodiments, as shown in FIG. 10, the device further includes a drug-free layer 1024 disposed on an outer surface of the annular body 1006, opposite the lumen 1012. For example, the drug-free layer may be configured to prevent direct contact between the matrix system or polymer of the annular body and surrounding tissue during in vivo use of the device. The API-free layer may have a tubular or non-tubular shape and may be formed of a matrix material such as silicone or other non-degradable or degradable polymers.

In one embodiment, the non-medicated or API-free (drug-free) layer 1024 is disposed outside of the API incorporated matrix 1006. In this embodiment, the API and the pH modifier 1014 can diffuse through the API-free layer 1024. In such embodiments, upon implantation of the drug delivery device, the API-free layer can help prevent direct contact between the API incorporated matrix and surrounding tissues, which may otherwise be irritated by a locally high concentration of the API, especially if the API is highly potent. In addition, if the API and the matrix polymer of the API incorporated matrix are not well mixed and clumps of the API are present near or on the surface of the API incorporated matrix, there is a risk of such API clumps being separated from the matrix by mechanical stress, swelling, and/or hydration of the matrix during in vivo use. The presence of the API-free layer outside of the API incorporated matrix can mitigate or eliminate these risks.

In one embodiment, as shown in FIG. 11, the device includes a rate controlling layer 1120 inside the lumen 1112 and also includes a non-medicated or API-free (drug-free) layer 1124 disposed outside of the API incorporated matrix 1106. In this embodiment, the API and the pH modifier 1114 can diffuse through the API-free layer. The presence of the API-free layer outside of the API incorporated matrix can mitigate or eliminate the above-described risks of direct contact between the API incorporated matrix and surrounding tissues as well as separation of any API clumps.

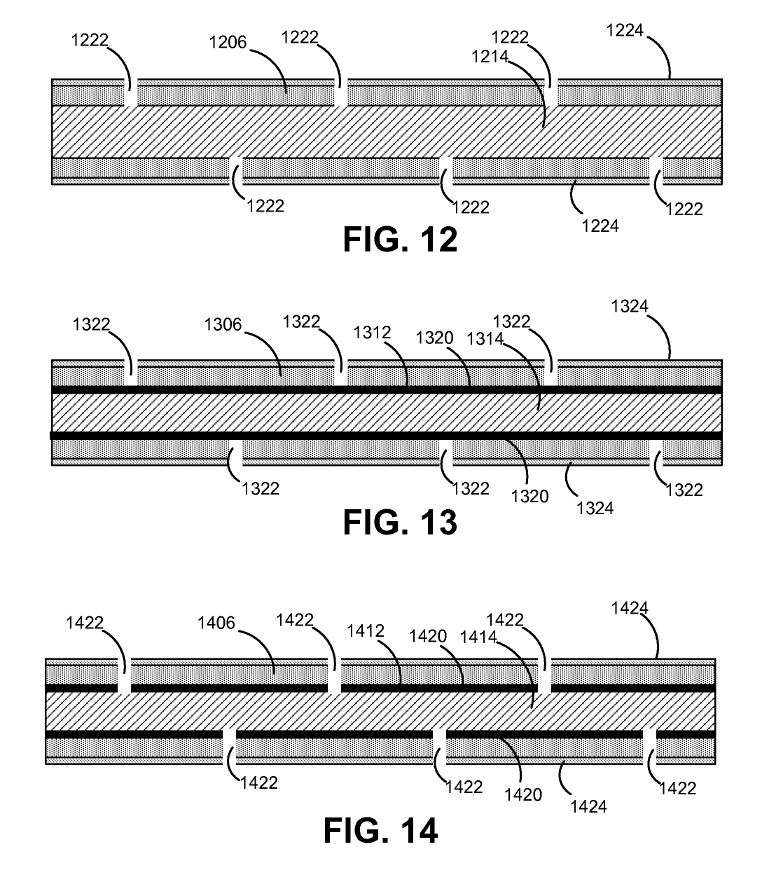

In one embodiment, as shown in FIG. 12, the device includes a non-medicated or API-free (drug-free) layer 1224 disposed outside of the API incorporated matrix 1206. In this embodiment, the pH modifier 1214 cannot diffuse through the wall of the API incorporated matrix 1206 or the wall of the API-free layer 1224, either at all or at an effective rate. In this case, there are multiple through-holes 1222 extending through the walls of both the API incorporated matrix 1206 and the API-free layer 1224. The number, size and position/layout of the holes will be determined by the pH altering strength of pH modifier, external media pH range to be adjusted, and/or API release rate from the API incorporated matrix. In one embodiment, both the API incorporated matrix and the API-free layer are formed of a silicone elastomer, and the holes are formed by precision cutter punches prior to insertion of the pH modifier tablet. As in previously described embodiments, the presence of the API-free layer outside of the API incorporated matrix can mitigate or eliminate the above described risks of direct contact between the API incorporated matrix and surrounding tissues as well as separation of any API clumps.

In one embodiment, as shown in FIG. 13, the device includes a rate controlling layer 1320 inside the lumen 1312 and also includes a non-medicated or API-free (drug-free) layer 1324 disposed outside of the API incorporated matrix. In this embodiment, the pH modifier 1314 can diffuse through the rate controlling layer 1320, but cannot diffuse through the tubular matrix wall 1306 of the API incorporated matrix or the wall of the API-free layer 1324, either at all or at an effective rate. In this case, there are multiple through-holes 1322 extending through the walls of both the API incorporated matrix 1306 and the API-free layer 1324, but not through the rate controlling layer 1320.

In one embodiment, as shown in FIG. 14, the device includes a rate controlling layer 1420 inside the lumen 1412 and also includes a non-medicated or API-free (drug-free) layer 1424 disposed outside of the API incorporated matrix. In this embodiment, the pH modifier 1414 cannot diffuse through the rate controlling layer 1420, through the tubular matrix wall 1406 of the API incorporated matrix, or through the wall of the API-free layer 1424, either at all or at an effective rate. In this case, there are multiple through-holes 1422 extending through the walls of both the API incorporated matrix 1406 and the API-free layer 1424, as well as through the rate controlling layer 1420.

Device Retention & Other Features

In certain embodiments, as shown in FIGS. 1A-1C, the device 100 includes a retention portion 104 that is associated with the drug release portion 102. In one embodiment, the device 100 includes an elastic retention frame 118 that is associated with the elongated body 106. For example the elastic retention frame 118 may be disposed in a retention frame lumen 116. In one embodiment, the elastic retention frame 118 is effective to bias the elongated body 106 into a coiled retention shape, as shown in FIG. 1A.

Thus, in some embodiments, the drug delivery devices have a structure that is deformable between the retention shape and a low profile shape for deployment, implantation, or insertion into a patient. The retention frame portion permits retaining the drug portion in the body, such as in the bladder. The retention frame portion may include a retention frame that is operable to impart a retention shape to the device structure, and is deformable between a relatively expanded shape and a relatively lower-profile shape.

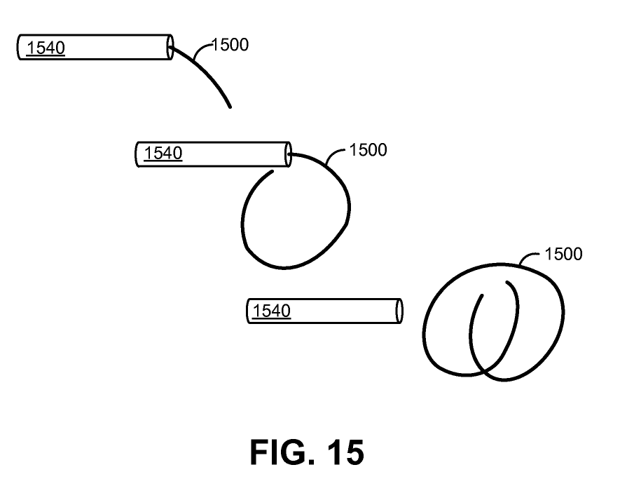

For example, as illustrated by FIG. 15, the retention frame may cause the device 1500 to naturally assume the relatively expanded shape, may be manipulated into the relatively lower-profile shape for insertion into the body, such as through implantation device 1540, and may spontaneously return to the relatively expanded shape upon insertion into the body. The retention frame in the relatively expanded shape may be shaped for retention in a body cavity, and the retention frame in the relatively lower-profile shape may be shaped for insertion into the body through the working channel of a deployment instrument such as a catheter or cystoscope. To achieve such a result, the retention frame may have an elastic limit, modulus, and/or spring constant selected to impede the device from assuming the relatively lower-profile shape once implanted, inserted, or deployed. Such a configuration may limit or prevent accidental expulsion of the device from the body under expected forces. For example, the device may be retained in the bladder during urination or contraction of the detrusor muscle.

In some embodiments, as shown in FIGS. 1A-1C, the device 100 includes a retention frame lumen 116, with an elastic retention frame 118 positioned in the retention frame lumen 116. In one embodiment, the retention frame includes or consists of an elastic wire. The elastic wire may be formed from a superelastic alloy, such as nitinol or another superelastic alloy. In another embodiment, the retention frame includes or consists of a cured polymer having a suitable durometer or flexibility, such as a high durometer silicone.

When the retention frame is in the relatively expanded shape, such as a coiled shape, the device may occupy a space having dimensions suited to impede expulsion from the bladder. When the retention frame is in the relatively lower-profile shape, the device may occupy an area suited for insertion into the body, such as through the working channel of a deployment instrument. The properties of the elastic wire cause the device to function as a spring, deforming in response to a compressive load but spontaneously returning to its initial shape once the load is removed.

The retention frame may be in a form having a high enough spring constant to retain the device within a body cavity, such as the bladder. A high modulus material may be used, or a low modulus material. Especially when a low-modulus material is used, the retention frame may have a diameter and/or shape that provide a spring constant without which the frame would significantly deform under the forces of urination. For example, the retention frame may include one or more windings, coils, spirals, or combinations thereof, specifically designed to achieve a desirable spring constant, such as a spring constant in the range of about 3 N/m to about 60 N/m, or more particularly, in the range of about 3.6 N/m to about 3.8 N/m. Such a spring constant may be achieved by one or more of the following techniques: increasing the diameter of the elastic wire used to form the frame, increasing the curvature of one or more windings of the elastic wire, and adding additional windings to the elastic wire. The windings, coils, or spirals of the frame may have a number of configurations. For example, the frame may be in a curl configuration having one or more loops, curls or sub-circles. The ends of the elastic wire may be adapted to avoid tissue irritation and scarring, such as by being soft, blunt, inwardly directed, joined together, or a combination thereof.

A retention frame that assumes a pretzel shape may be relatively resistant to compressive forces. The pretzel shape essentially comprises two sub-circles, each having its own smaller arch and sharing a common larger arch. When the pretzel shape is first compressed, the larger arch absorbs the majority of the compressive force and begins deforming, but with continued compression the smaller arches overlap, and subsequently, all three of the arches resist the compressive force. The resistance to compression of the device as a whole increases once the two sub-circles overlap, impeding collapse and voiding of the device as the bladder contracts during urination.

The retention frame may have a two-dimensional structure that is confined to a plane, a three-dimensional structure, such as a structure that occupies the interior of a spheroid, or some combination thereof.

Applications and Methods of Using Drug Delivery Devices

The drug delivery devices described herein can be adapted for use for a variety of different drugs for a variety of different indications, treatments, therapies, and the like.

The drug can include essentially any therapeutic, prophylactic, or diagnostic agent, such as one that would be useful to deliver locally or regionally to a desired tissue site. For example, the tissue site may include the urinary bladder, the prostate, etc. As used herein, the term “drug” with reference to any specific drug described herein includes its alternative forms, such as salt forms, free acid forms, free base forms, and hydrates. The drug may be a small molecule drug or a biologic. The drug may be a metabolite. Pharmaceutically acceptable excipients are known in the art and may include lubricants, viscosity modifiers, surface active agents, osmotic agents, diluents, and other non-active ingredients of the formulation intended to facilitate handling, stability, dispersibility, wettability, and/or release kinetics of the drug.

For example, in one embodiment, the drug delivery device is configured to treat urinary incontinence, frequency, or urgency, including urge incontinence and neurogenic incontinence, as well as trigonitis. Drugs that may be used include anticholinergic agents, antispasmodic agents, anti-muscarinic agents, β-2 agonists, alpha adrenergics, anticonvulsants, norepinephrine uptake inhibitors, serotonin uptake inhibitors, calcium channel blockers, potassium channel openers, and muscle relaxants. Representative examples of suitable drugs for the treatment of incontinence include oxybutynin, S-oxybutytin, emepronium, verapamil, imipramine, flavoxate, atropine, propantheline, tolterodine, rociverine, clenbuterol, darifenacin, terodiline, trospium, hyoscyamin, propiverine, desmopressin, vamicamide, clidinium bromide, dicyclomine HCl, glycopyrrolate aminoalcohol ester, ipratropium bromide, mepenzolate bromide, methscopolamine bromide, scopolamine hydrobromide, iotropium bromide, fesoterodine fumarate, YM-46303 (Yamanouchi Co., Japan), lanperisone (Nippon Kayaku Co., Japan), inaperisone, NS-21 (Nippon Shinyaku Orion, Formenti, Japan/Italy), NC-1800 (Nippon Chemiphar Co., Japan), Z D-6169 (Zeneca Co., United Kingdom), and stilonium iodide.

In another embodiment, the drug delivery device is configured to treat urinary tract cancer, such as bladder cancer and prostate cancer. Drugs that may be used include antiproliferative agents, cytotoxic agents, chemotherapeutic agents, or a combination thereof. Representative examples of drugs which may be suitable for the treatment of urinary tract cancer include Bacillus Calmette Guerin (BCG) vaccine, cisplatin, doxorubicin, valrubicin, gemcitabine, mycobacterial cell wall-DNA complex (MCC), methotrexate, vinblastine, thiotepa, mitomycin, fluorouracil, leuprolide, diethylstilbestrol, estramustine, megestrol acetate, cyproterone, flutamide, a selective estrogen receptor modulators (i.e. a SERM, such as tamoxifen), botulinum toxins, and cyclophosphamide. The drug may be a biologic, and it may comprise a monoclonal antibody, a TNF inhibitor, an anti-leukin, or the like. The drug also may be an immunomodulator, such as a TLR agonist, including imiquimod or another TLR7 agonist. The drug also may be a kinase inhibitor, such as a fibroblast growth factor receptor-3 (FGFR3)-selective tyrosine kinase inhibitor, a phosphatidylinositol 3 kinase (PI3K) inhibitor, or a mitogen-activated protein kinase (MAPK) inhibitor, among others or combinations thereof. Other examples include celecoxib, erolotinib, gefitinib, paclitaxel, polyphenon E, valrubicin, neocarzinostatin, apaziquone, Belinostat, Ingenol mebutate, Urocidin (MCC), Proxinium (VB 4845), BC 819 (BioCancell Therapeutics), Keyhole limpet haemocyanin, LOR 2040 (Lorus Therapeutics), urocanic acid, OGX 427 (OncoGenex), and SCH 721015 (Schering-Plough). The drug treatment may be coupled with a conventional radiation or surgical therapy targeted to the cancerous tissue.

Other intravesical cancer treatments include small molecules, such as Apaziquone, adriamycin, AD-32, doxorubicin, doxetaxel, epirubicin, gemcitabine, HTI-286 (hemiasterlin analogue), idarubicin, γ-linolenic acid, mitozantrone, meglumine, and thiotepa; large molecules, such as Activated macrophages, activated T cells, EGF-dextran, HPC-doxorubicin, IL-12, IFN-a2b, IFN-γ, α-lactalbumin, p53 adenovector, TNFα; combinations, such as Epirubicin+BCG, IFN+farmarubicin, Doxorubicin+5-FU (oral), BCG+IFN, and Pertussis toxin+cystectomy; activated cells, such as macrophages and T cells; intravesical infusions such as IL-2 and Doxorubicin; chemosensitizers, such as BCG+antifirinolytics (p aramethylbenzoic acid or aminocaproic acid) and Doxorubicin+verapimil; diagnostic/imaging agents, such as Hexylaminolevulinate, 5-aminolevulinic acid, Iododexyuridine, HMFG1 Mab+Tc99m; and agents for the management of local toxicity, such as Formaline (hemorrhagic cystitis).

In yet another embodiment, the drug delivery device is configured to treat infections involving the bladder, the prostate, and the urethra. Antibiotics, antibacterial, antifungal, antiprotozoal, antiseptic, antiviral and other antiinfective agents can be administered for treatment of such infections. Representative examples of drugs for the treatment of infections include mitomycin, ciprofloxacin, norfloxacin, ofloxacin, methanamine, nitrofurantoin, ampicillin, amoxicillin, nafcillin, trimethoprim, sulfonamides trimethoprimsulfamethoxazole, erythromycin, doxycycline, metronidazole, tetracycline, kanamycin, penicillins, cephalosporins, and aminoglycosides.

In still another embodiment, the drug delivery device is configured to treat fibrosis of a genitourinary site, such as the bladder or uterus. Representative examples of drugs for the treatment of fibroids include pentoxphylline (xanthine analogue), antiTNF, antiTGF agents, GnRH analogues, exogenous progestins, antiprogestins, selective estrogen receptor modulators, danazol and NSAIDs.

In another embodiment, the drug delivery device is configured treat neurogenic bladder. Representative examples of drugs for the treatment of neurogenic bladder include analgesics or anaesthetics, such as lidocaine, bupivacaine, mepivacaine, prilocaine, articaine, and ropivacaine; anticholinergics; antimuscarinics such as oxybutynin or propiverine; a vanilloid, such as capsaicin or resiniferatoxin; antimuscarinics such as ones that act on the M3 muscarinic acetylcholine receptor (mAChRs); antispasmodics including GABA_{B} agonists such as baclofen; botulinum toxins; capsaicins; alpha-adrenergic antagonists; anticonvulsants; serotonin reuptake inhibitors such as amitriptyline; and nerve growth factor antagonists. In various embodiments, the drug may be one that acts on bladder afferents or one that acts on the efferent cholinergic transmission, as described in Reitz et al., Spinal Cord 42:267-72 (2004).

In a further embodiment, the drug is selected from those known for the treatment of incontinence due to neurologic detrusor overactivity and/or low compliant detrusor. Examples of these types of drugs include bladder relaxant drugs (e.g., oxybutynin (antimuscarinic agent with a pronounced muscle relaxant activity and local anesthetic activity), propiverine, impratroprium, tiotropium, trospium, terodiline, tolterodine, propantheline, oxyphencyclimine, flavoxate, and tricyclic antidepressants; drugs for blocking nerves innervating the bladder and urethra (e.g., vanilloids (capsaicin, resiniferatoxin), botulinum-A toxin); or drugs that modulate detrusor contraction strength, micturition reflex, detrusor sphincter dyssynergia (e.g., GABAb agonists (baclofen), benzodiazapines). In another embodiment, the drug is selected from those known for the treatment of incontinence due to neurologic sphincter deficiency. Examples of these drugs include alpha adrenergic agonists, estrogens, beta-adrenergic agonists, tricyclic antidepressants (imipramine, amitriptyline). In still another embodiment, the drug is selected from those known for facilitating bladder emptying (e.g., alpha adrenergic antagonists (phentolamine) or cholinergics). In yet another embodiment, the drug is selected from among anticholinergic drugs (e.g., dicyclomine), calcium channel blockers (e.g., verapamil) tropane alkaloids (e.g., atropine, scopolamine), nociceptin/orphanin FQ, and bethanechol (e.g., m3 muscarinc agonist, choline ester).

In another embodiment, the drug delivery device is configured to treat pain in a patient. A variety of anesthetic agents, analgesic agents, and combinations thereof may be used. In embodiments, the device delivers one or more anesthetic agents. The anesthetic agent may be a cocaine analogue. In embodiments, the anesthetic agent is an aminoamide, an aminoester, or combinations thereof. Representative examples of aminoamides or amide-class anesthetics include articaine, bupivacaine, carticaine, cinchocaine, etidocaine, levobupivacaine, lidocaine, mepivacaine, prilocaine, ropivacaine, and trimecaine. Representative examples of aminoesters or ester-class anesthetics include amylocaine, benzocaine, butacaine, chloroprocaine, cocaine, cyclomethycaine, dimethocaine, hexylcaine, larocaine, meprylcaine, metabutoxycaine, orthocaine, piperocaine, procaine, proparacaine, propoxycaine, proxymetacaine, risocaine, and tetracaine. Other anesthetics, such as lontocaine, also may be used. The drug also can be an antimuscarinic compound that exhibits an anesthetic effect, such as oxybutynin or propiverine. In one embodiment, the analgesic agent includes an opioid. Representative examples of opioid agonists include alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts thereof, and mixtures thereof. Other opioid drugs, such as mu, kappa, delta, and nociception opioid receptor agonists, are contemplated. Representative examples of other suitable pain relieving agents include salicyl alcohol, phenazopyridine hydrochloride, acetaminophen, acetylsalicylic acid, flufenisal, ibuprofen, indoprofen, indomethacin, and naproxen.

In some embodiments, the drug delivery device is configured to treat inflammatory conditions such as interstitial cystitis, radiation cystitis, painful bladder syndrome, prostatitis, urethritis, post-surgical pain, and kidney stones. Non-limiting examples of specific drugs for these conditions include lidocaine, glycosaminoglycans (e.g., chondroitin sulfate, sulodexide), pentosan polysulfate sodium (PPS), dimethyl sulfoxide (DMSO), oxybutynin, mitomycin C, heparin, flavoxate, ketorolac, or a combination thereof. For kidney stones, the drug(s) may be selected to treat pain and/or to promote dissolution of renal stones. Other non-limiting examples of drugs that may be used in the treatment of IC include nerve growth factor monoclonal antibody (MAB) antagonists, such as Tanezumab, and calcium channel alpha-2-delta modulators, such as PD-299685 or gabepentin.

In certain embodiments, methods of administering a drug to a patient in need thereof include inserting into the patient a drug delivery device as described herein and releasing the drug and pH modifier therefrom by diffusion. In certain embodiments, the pH modifier is released in an amount effective to cause a proximal media pH adjacent an outer surface of the annular body to be dominated by a microenvironment pH created by the diffusion of the pH modifier and not by a macro pH of an external media.

In one embodiment, the device is inserted into the urinary bladder of the patient via the patient's urethra. In one embodiment, the device includes an elastic retention frame associated with the elongated body, such as described above, and inserting the device into the patient comprises deforming the device into a relatively straightened shape and inserting the device through a lumen into a body cavity of a patient, such that the device returns to the retention shape within the body cavity (as shown in FIG. 15).

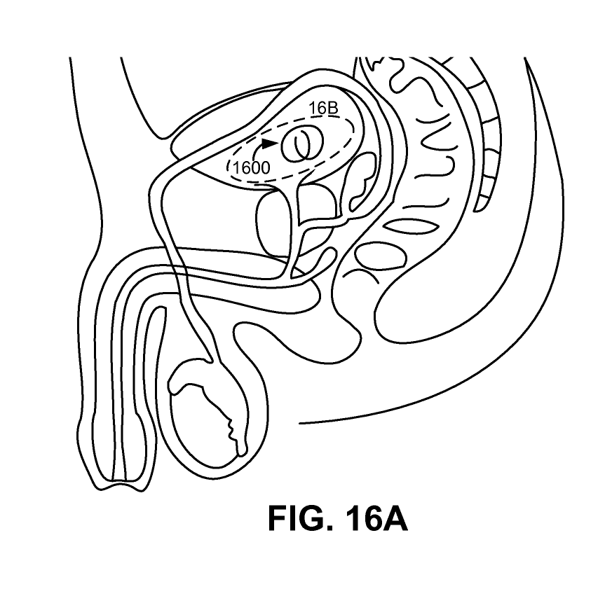

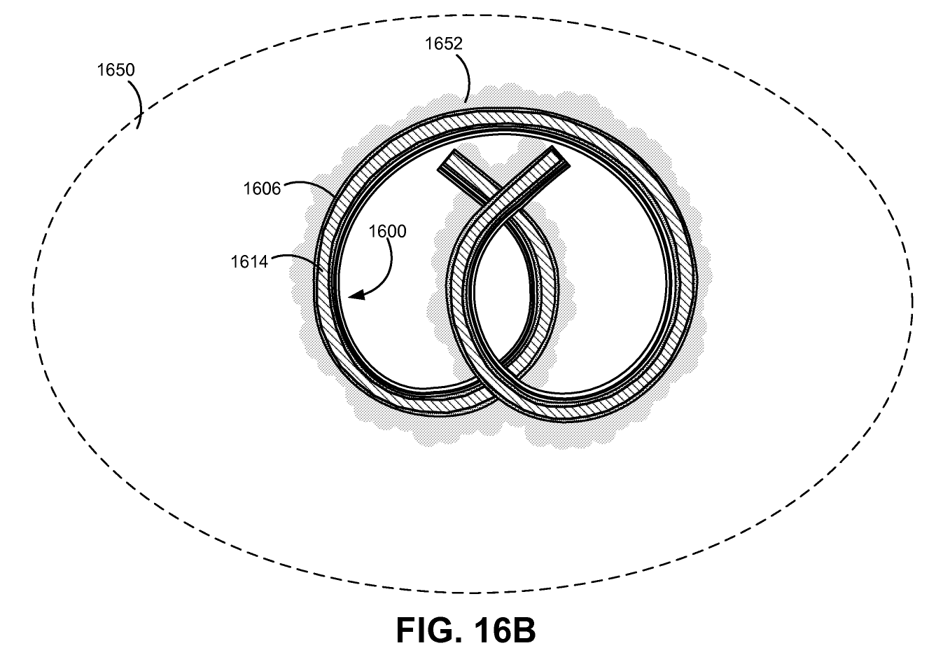

Once in the bladder, as shown in FIGS. 16A-16B, the device 1600 contacts biological fluid at the site (e.g., urine) and the pH modifier 1614 becomes solubilized and released from the device, such that a proximal media pH adjacent an outer surface of the annular body 1606 is dominated by a microenvironment pH 1652 created by the diffusion of the pH modifier 1614 and not by a macro pH of the external media 1650. In certain embodiments, the release of the pH modifier from the device creates an overall surface pH modification at the outer surface of the device.

In one embodiment, the drug delivery device is used in association with the placement of a ureteral stent, such as to treat pain, urinary urgency or urinary frequency resulting from ureteral stent placement. Non-limiting examples of specific drugs for such treatment include anti-muscarinics, beta-blockers, narcotics, and phenazopyridine, among others.

The device may include a retrieval string to facilitate withdrawal of a device from the patient. For example, the retrieval string may extend (or be selectively extendable) from the patient's urethra to facilitate manual removal of the device residing the patient's bladder.

In one embodiment, the device includes at least one radio-opaque portion or structure to facilitate detection or viewing (e.g., by X-ray imaging or fluoroscopy) of the device by a medical practitioner as part of the implantation, insertion, or retrieval procedure. In one embodiment, the device is constructed, at least in part, of a material that includes a radio-opaque filler material, such as barium sulfate or another radio-opaque material known in the art. Fluoroscopy may be used during deployment and/or retrieval of the device by providing accurate real-time imaging of the position and orientation of the device to the practitioner performing the procedure.

The device may be implanted, inserted, or deployed at any desired site, including in the urinary bladder or other body cavity or lumen of a patient in need thereof. The drug delivery devices provided herein also may be configured for subcutaneous, intramuscular, intraocular, intraperitoneal, and/or intrauterine implantation. Subsequently, the device may release one or more drugs for the treatment of one or more conditions, locally to one or more tissues at the deployment site and/or regionally to other tissues distal from the deployment site. The release may be controlled over an extended period. Thereafter, the device may be retrieved, resorbed, excreted, or some combination thereof.

In one example, the device is inserted into a patient by passing the drug delivery device through a deployment instrument and releasing the device from the deployment instrument into the body. In cases in which the device is inserted into a body cavity such as the bladder, the device assumes a retention shape, such as an expanded or higher profile shape, once the device emerges from the deployment instrument into the cavity.

Once inserted, the device may release the drug. The device may provide extended, continuous, intermittent, or periodic release of a desired quantity of drug over a desired, predetermined time period. In embodiments, the device can deliver the desired dose of drug over an extended period, such as 12 hours, 24 hours, 5 days, 7 days, 10 days, 14 days, or 20, 25, 30, 45, 60, or 90 days, or more. In one embodiment, the drug is released continuously over a period of from about 1 day to about 45 days in a therapeutically effective amount. The rate of delivery and dosage of the drug can be selected depending upon the drug being delivered and the disease or condition being treated.

The device may be deployed into the bladder of a patient in an independent procedure or in conjunction with another urological or other procedure or surgery, either before, during, or after the other procedure. The device may release one or more drugs that are delivered to local and/or regional tissues for therapy or prophylaxis, either peri-operatively, post-operatively, or both.

In an embodiment, the device is configured for intravesical insertion for use in the local administration of one or more drugs into the bladder to treat interstitial cystitis, radiation cystitis, pelvic pain, overactive bladder syndrome, bladder cancer, neurogenic bladder, neuropathic or non-neuropathic bladder-sphincter dysfunction, infection, post-surgical pain or other diseases, disorders, and conditions treated with drugs delivered to the bladder. The device may deliver drugs that improve bladder function, such as bladder capacity, compliance, and/or frequency of uninhibited contractions, that reduce pain and discomfort in the bladder or other nearby areas, or that have other effects, or combinations thereof. The bladder-deployed device also may deliver a therapeutically effective amount of one or more drugs to other genitourinary sites within the body, such as other locations within urological or reproductive systems of the body, including one or both of the kidneys, the urethra, one or both of the ureters, the penis, the testes, one or both of the seminal vesicles, one or both of the vas deferens, one or both of the ejaculatory ducts, the prostate, the vagina, the uterus, one or both of the ovaries, or one or both of the fallopian tubes, among others or combinations thereof. For example, the intravesical drug delivery device may be used in the treatment of kidney stones or fibrosis, erectile dysfunction, among other diseases, disorders, and conditions.

In some embodiments, the drug delivery device is deployed into the bladder of a patient for regional drug delivery to one or more nearby genitourinary sites. The device may release drug locally to the bladder and regionally to other sites near the bladder. Such delivery may provide an alternative to systemic administration, which may entail undesirable side effects or result in insufficient bioavailability of the drug.

Methods of Manufacturing Drug Delivery Devices

Methods of manufacturing the drug delivery devices described herein are also provided. In certain embodiments, the optional API-free layer is first formed, such as by molding, extrusion, or other processes. The API-free layer may be formed as a tubular body including at least one lumen extending therethrough. For example, the API-free layer may be formed as a dual lumen tube including a first lumen for receiving the API incorporated matrix and the pH modifier and a second lumen for receiving a retention frame. Next, a measured amount of the API incorporated matrix in an uncured state is injected or otherwise directed into the first lumen. The API-free layer is positioned between a pair of rollers or other compression mechanism configured to compresses the API-free layer. The API-free layer is passed through the pair of rollers or the pair of rollers is passed along the API-free layer, such that the uncured API incorporated matrix is distributed relatively evenly over the inner surface of the API-free layer. In this manner, the API incorporated matrix forms a generally tubular shape within the API-free layer. Then the API-free layer is allowed to relax, returning to its original shape, and the API incorporated matrix is allowed to cure. An optional rate controlling layer may be introduced inside the API incorporated matrix in a similar fashion. Next, the pH modifier is inserted within the cured API incorporated matrix (or optional rate controlling layer). The pH modifier may be formed as tablet, although other configurations may be used. Finally, the terminal ends of the delivery device may be sealed. According to other example methods of forming the drug delivery device, the API incorporated matrix is molded within the API-free layer or is molded and then inserted within the API-free layer (or no API-free layer is used).

These methods may be adapted for embodiments in which the annular body is a biocompatible polymer that is formed in a similar way to the API-free layer and then filled with the optional rate controlling layer and pH modifier and drug.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

1. A drug delivery device comprising:
an elongated annular body formed of a matrix system comprising a drug dispersed in a biocompatible polymer, the elongated body having a first end, an opposed second end, and a lumen extending between the first and second ends; and
a pH modifier disposed in the lumen,
wherein the device is configured to release in vivo (i) the drug by diffusion from the annular body and (ii) the pH modifier by diffusion from the lumen through the annular body.

2. The device of claim 1, wherein the pH modifier comprises a carbonate or a citrate.

3. The device of claim 1, which comprises one or more mechanically formed through-holes through the annular body.

4. The device of claim 1, wherein the pH modifier is in the form of a plurality of tablets.

5. The device of claim 1, further comprising a rate controlling layer disposed in the lumen between the pH modifier and the matrix system.

6. The device of claim 5, wherein the rate controlling layer comprises a polymer.

7. The device of claim 5, wherein the rate controlling layer comprises a porous material.

8. The device of claim 3, which comprises one or more mechanically formed through-holes through both the annular body and the rate controlling layer.

9. The device of claim 1, wherein the device is configured to release the pH modifier in an amount effective to cause a proximal media pH adjacent an outer surface of the annular body to be dominated by a microenvironment pH created by the diffusion of the pH modifier and not by a macro pH of an external media.

10. The device of claim 1, further comprising an elastic retention frame associated with the elongated body and effective to bias the elongated body into a coiled retention shape.

11. The device of claim 10, wherein the elastic retention frame comprises:
a nitinol or other superelastic wire, or
a high durometer silicone.

12. The device of claim 1, wherein the biocompatible polymer is a low durometer silicone.

13. The device of claim 1, wherein the drug comprises between 0.1 percent and 40 percent, by weight, of the matrix system.

14. The device of claim 1, further comprising a drug-free layer disposed on an outer surface of the matrix system opposite the lumen.

15. The device of claim 14, wherein the drug-free layer is configured to prevent direct contact between the matrix system of the annular body and surrounding tissue during in vivo use of the device.

16. A drug delivery device comprising:
an elongated annular body formed of a biocompatible polymer, the elongated body having a first end, an opposed second end, and a lumen extending between the first and second ends; and
a drug and a pH modifier disposed in separate locations in the lumen,
wherein the device is configured to release in vivo the drug and the pH modifier by diffusion from the lumen through the annular body, and
wherein the device is configured to release the pH modifier in an amount effective to cause a proximal media pH adjacent an outer surface of the annular body to be dominated by a microenvironment pH created by the diffusion of the pH modifier and not by a macro pH of an external media.

17. The device of claim 16, wherein the pH modifier comprises a carbonate or a citrate.

18. The device of claim 16, which comprises one or more mechanically formed through-holes through the annular body.

19. The device of claim 16, wherein the pH modifier, the drug, or both, is in the form of a plurality of tablets.

20. The device of claim 16, further comprising an elastic retention frame associated with the elongated body and effective to bias the elongated body into a coiled retention shape.

21. The device of claim 20, wherein the elastic retention frame comprises:
a nitinol or other superelastic wire, or
a high durometer silicone.

22. The device of claim 16, wherein the biocompatible polymer is a low durometer silicone.

23. The device of claim 16, further comprising a rate controlling layer disposed in the lumen between the biocompatible polymer and the drug and pH modifier.

24. The device of claim 23, wherein the rate controlling layer comprises a polymer.

25. The device of claim 23, wherein the rate controlling layer comprises a porous material.

26. The device of claim 23, which comprises one or more mechanically formed through-holes through both the annular body and the rate controlling layer.

27. A method of administering a drug to a patient in need thereof, comprising:
inserting into the patient a device comprising:
an elongated annular body formed of a matrix system comprising a drug dispersed in a biocompatible polymer, the elongated body having a first end, an opposed second end, and a lumen extending between the first and second ends; and
a pH modifier disposed in the lumen;
releasing the drug by diffusion from the annular body to the body of the patient; and
releasing the pH modifier by diffusion from the lumen through the annular body to the body of the patient.

28-36. (canceled)

37. The method of claim 27, wherein the pH modifier is released in an amount effective to cause a proximal media pH adjacent an outer surface of the annular body to be dominated by a microenvironment pH created by the diffusion of the pH modifier and not by a macro pH of an external media.

38-40. (canceled)

41. A method of administering a drug to a patient in need thereof, comprising:
inserting into the patient a device comprising:
an elongated annular body formed of a biocompatible polymer, the elongated body having a first end, an opposed second end, and a lumen extending between the first and second ends; and
a drug and a pH modifier disposed in separate locations in the lumen; and
releasing the drug and the pH modifier by diffusion from the lumen through the annular body,
wherein the pH modifier is released in an amount effective to cause a proximal media pH adjacent an outer surface of the annular body to be dominated by a microenvironment pH created by the diffusion of the pH modifier and not by a macro pH of an external media.

42-50. (canceled)
